(19) United States
(12) Patent Application Publication
(10) Pub. No.: US 2023/0277129 A1
(43) Pub. Date: Sep. 7, 2023

(54) IMPACT DETECTION DEVICES AND SYSTEMS

(71) Applicant: Apple Inc., Cupertino, CA (US)

(72) Inventors: Kathleen A. Bergeron, Los Gatos, CA (US); Edward Siahaan, San Francisco, CA (US); Esge B. Andersen, Campbell, CA (US); Geng Luo, Santa Clara, CA (US)

(21) Appl. No.: 17/816,933

(22) Filed: Aug. 2, 2022

Related U.S. Application Data
(60) Provisional application No. 63/267,528, filed on Feb. 3, 2022.

(51) Int. Cl. A61B 5/00; G08B 21/04; A61B 5/11; H04R 1/10

(52) U.S. Cl. CPC A61B 5/6803; A61B 5/1117; G08B 21/043; G08B 21/0446; H04R 1/1091; A61B 2562/0219; A63B 2220/40; A63B 2220/803

(57) ABSTRACT

A impact detection system can include an earbud having a first motion sensor and a wearable electronic device in electrical communication with the earbud and comprising a second motion sensor electrically connected to a processor. The processor can determine if a threshold event has occurred based on a first motion detected by the first motion sensor and a second motion detected by the second motion sensor.

### CROSS-REFERENCE TO RELATED APPLICATION(S)

This application claims the benefit of U.S. Provisional Pat. Application No. 63/267,528, filed 3 Feb. 2022, and entitled “IMPACT DETECTION DEVICES AND SYSTEMS,” the disclosure of which is incorporated herein by reference in its entirety.

### FIELD

The described embodiments relate generally to impact detection systems. More particularly, the present embodiments relate to portable and wearable impact detection devices, systems, and methods.

### BACKGROUND

Recent advances in portable computing have enabled wearable electronic devices capable of detecting when a user falls or experiences accelerations indicative of a fall or an impact. However, the ability of a wearable electronic devices to detect such impact events is limited to the data that can be gathered by the device. A wearable device such as an electronic watch, for example, can detect motion at the user’s wrist, but is generally limited to information at that location on the user’s body. The user can experience detrimental injuries resulting from falls or impacts, including injuries to the head or neck. Electronic devices that are normally worn by users lack the necessary components and functionalities necessary to accurately and confidently detect these falls and impact events.

Thus, what is needed in the art are systems, methods, and devices for accurately and confidently detecting when a user experiences an impact exceeding a threshold acceleration, event, or motion in a known, specific location.

### SUMMARY

In one example of the present disclosure, a portable impact detection device can include a first motion sensor to gather first motion data associated with the portable impact detection device, a processor electrically coupled to the first motion sensor, and a receiver configured to receive second motion data from a head-mountable device. The processor determines that a threshold motion of the head-mountable device has occurred based on the first motion data and the second motion data.

In one example of the portable impact detection device, the head-mountable device can include an earbud. The earbud can include a second motion sensor, an antenna, and a second processor electrically connected to the second motion sensor and the antenna. The second processor can identify a threshold motion detected by the second motion sensor and cause the antenna to send a signal that includes the second motion data. In one example, the signal is sent to the portable concussion detection device. In one example, the portable concussion detection device can include a wearable electronic device. In one example, the wearable electronic device comprises a smartwatch. In one example, the wearable electronic device can include a smartphone.

In one example of the present disclosure, an impact detection system can include an earbud having a first motion sensor and a wearable electronic device in electrical communication with the earbud. The wearable electronic device can include a second motion sensor electrically connected to a processor. The processor can determine if a threshold event has occurred based on a first motion detected by the first motion sensor and a second motion detected by the second motion sensor.

In one example of the impact detection system, the earbud can further include an antenna electrically connected to the first motion sensor, the antenna configured to transmit data associated with the first motion to the wearable electronic device. In one example, the earbud further includes an optical sensor configured to detect when the earbud is mounted to a user. In one example, the processor causes the antenna to transmit the data if the optical sensor detects the earbud mounted to the user. In one example, the data indicates an acceleration of a head of a user to which the earbud is mounted. In one example, the wearable electronic device includes an electronic watch. In one example, the wearable electronic device comprises a smartphone. In one example of the impact detection system, the head-mountable device is a first head-mountable device and the impact detection system further includes a second head-mountable device having a third motion sensor. In such an example, the processor determines if a threshold event has occurred based on a first motion detected by the first motion sensor, a second motion detected by the second motion sensor, and a third motion detected by the third motion sensor.

In one example of the present disclosure, a method of detecting a threshold motion can include detecting a motion of a head-mountable device, sending a signal from the head-mountable device to an external device, and determining that a head to which the head-mountable device is mounted has experienced a threshold motion. The signal can indicate the motion and the determination can be based on the motion.

In one example of the method, the external device can include a wearable electronic device. In one example, the method can further include detecting a motion of the wearable electronic device. In one example, determining the head has experienced the threshold motion is based on the motion of the head-mountable device and the motion of the wearable electronic device. In one example, the method can include determining an acceleration of the head based on the detected motion of the head-mountable device. In one example, the method can include detecting when the head-mountable device is mounted to the head.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example of an impact detection system;

FIG. 2 shows an example of an earbud worn by a user;

FIG. 3 shows an example of a wearable electronic device worn by a user;

FIG. 4 shows an example of an impact detection system including an earbud and a wearable device worn by a user;

FIG. 5 shows an example of an impact detection system including an earbud and a wearable device worn by a user;

FIG. 6 shows a flowchart of a method of detecting an impact;

FIG. 7 shows a flowchart of a method of detecting an impact;

FIG. 8 shows a flowchart of a method of detecting an impact; and

FIG. 9 shows a flowchart of a method of detecting an impact.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to an impact detection system. In at least one example, an impact detection system can include an earbud having a first motion sensor and a wearable electronic device in electrical communication with the earbud and including a second motion sensor electrically connected to a processor. The processor can determine if an impact event has occurred based on a first motion detected by the first motion sensor and a second motion detected by the second motion sensor.

The terms “injurious impact” and “injurious motion,” or other similar or related terms, are used herein to denote accelerations and motion trajectories above a predetermined threshold that when experienced by a user or a body part of a user, for example the head of a user, may lead to or, be indicative of, an injury to that body part. These motions or events are also referred to herein as “threshold motions” or “threshold events.” That is, an “injurious” or “threshold” motion or event can be experienced by a device of the impact detection systems described herein but this does not necessarily mean the device itself has experienced injury or damage. Rather, an injurious motion can include a motion (such as an acceleration or motion path/position change) of a device connected to the body of the user that, when experienced by body part to which the device is connected, exceeds certain measurable thresholds such that the motion could be indicative of an injury to that body part. For example, a device of an impact detection system can be secured to the head of a user. If the user’s head is impacted during an activity, the device connected thereto will experience an acceleration similar to that experienced by the user’s head. It can be assumed that the head to which the device is secured may also have experienced the acceleration due to the impact. Accelerations above a certain threshold, for example a predetermined threshold level, can be indicative of a head injury, for example a concussion, or may be sufficiently severe to perform additional steps, investigations, and data gathering.

Thus, detecting the acceleration of the device can be extrapolated to the acceleration experienced by the head and a “threshold motion” can be determined or detected. Accordingly, as described herein, the devices and systems described herein can detect or experience “injurious” motions or impacts that refer to a possible injury of the user’s head or other body part to which the device is secured. Injurious motions or impacts may not always result in injury but can indicate that the experienced (and detected) motion or impact has risen above a threshold level that would normally be considered injurious or warrant further examination and investigation to determine if an injury has occurred.

An electronic device worn by a user can include a motion sensor that detects the motion of the device when the user is active. Users can wear electronic devices, such as electronic watches and smartphones, when exercising, hiking, biking, skiing, kickboxing, walking, or while doing any other physical activity. A user can wear an electronic watch while biking, for example, to track heart rate, calculate mileage and speed, and for navigating trails and roads. Motion sensors built into the electronic watch can incorporate one or more accelerometers and gyroscopes to detect whether or not the user has experienced a fall or other harmful impact to his or her body while biking. However, motions detected by the watch secured to the user’s arm or wrist are not always indicative of the motions and accelerations experienced by the user’s head during the activity, which may be of particular interest or concern seeing that head injuries can be especially long-term and/or life-altering in many cases.

For example, the electronic watch worn by a user striking a punching bag during a workout may experience and detect repeated accelerations above an injury threshold while the user’s head is not experiencing injurious accelerations. The same can be true of a user chopping wood or even skiing and running into a tree with his or her arm while the user’s head remains unscathed.

Accordingly, impact detection systems, devices, and methods described herein can include one or more additional devices, for example a head-mountable device, for detecting motions at the user’s head or other any other body part. In this way, motions and accelerations that correlate to multiple body parts of the user, including the user’s head, can be taken into account when one or more processors of the system determine whether or not the user has experienced an impact and whether that impact is above a certain injury threshold of motions and accelerations that could potentially cause bodily injury. The presence of multiple devices having motion sensors electrically communicating with one another within the same system can provide multiple data sources that increase the accuracy of impact event determinations and reduce false determinations. For example, while many of the examples and embodiments described herein describe a user wearing a single earbud including a motion sensor communicating with another electronic device, any of these examples can also include a user wearing two earbuds with respective sensors communicating with each other and another electronic device.

As noted above, in addition to a wearable electronic device worn by the user, an impact detection system can also include a head-mountable device such as one or more earbuds worn by the user. The wearable electronic device, for example an electronic watch, and the head-mountable device, for example an earbud, can be in electrical communication with each other within the system such that each device can transmit detected motions from one device to the other. In this way, the electronic watch, upon a detection of an injurious motion or event that includes an acceleration above a predetermined injury threshold, can receive data transmitted from the earbud of the system indicative of a motion experienced at the earbud. The motion of the earbud can be extrapolated to correlate to a motion of the user’s head on which the earbud is mounted. The earbud can include one or more motion sensors and both devices of the system can include one or more antennas that transmit/communicate motion data between devices.

One or more impact detection systems can include more than two devices such that more reference points of motion data can be detected and communicated for a more accurate determination based on more information. The systems described herein can also continue to monitor the user’s pulse, oxygen levels, and movements after an impact has occurred to determine whether the user is conscious. Furthermore, systems described herein can utilize one or more of the devices to interact with the user for alerting the user or requesting confirmation of a detected impact event. Based on the user’s response or lack thereof, the system can use one or more of the devices to communicate with emergency personnel to seek help if needed.

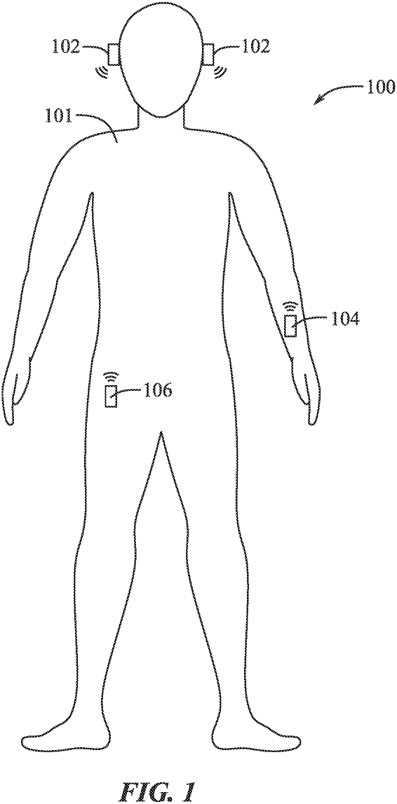

These and other embodiments are discussed below with reference to FIGS. 1-9. However, those skilled in the art will readily appreciate that the detailed description given herein with respect to these Figures is for explanatory purposes only and should not be construed as limiting. Furthermore, as used herein, a system, a method, an article, a component, a feature, or a sub-feature including at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates an example of an impact detection system 100 including a head-mountable device 102 mounted on the head of a user 101 and one or more other wearable electronic devices, including electronic watch 104 and smartphone 106. In at least one example, each device 102, 104, 106 can be in electrical communication with one or more other devices of the system 100 such that signals (indicated by curved wave lines next to each device in FIG. 1) can be sent and received between the head-mountable device 102 and any of the wearable electronic devices 104, 106.

In at least one example, the head-mountable device 102 can be wirelessly connected to one or more of the wearable electronic devices 104, 106. For example, the head-mountable device 102 can be in wireless communication with one or more of the wearable electronic devices 104, 106 such that the head-mountable device 102 can communicate with one or more of the wearable electronic devices 104, 106. As used herein, the terms “communicate,” “connected,” or other forms of these terms used with reference to various electronic devices of the systems described herein refers to the receiving and sending of signals from one device to another to receive and transfer data. Various forms of data discussed herein can contain information of various forms. Each of the devices described herein, including the head-mountable device 102 and wearable electronic devices 104, 106 can include one or more communication modules each having one or more antennas or other transmitter/receiver components for wireless communication and connectivity within the system 100.

For example, any of the devices 102, 104, 106 can include one or more antennas, Bluetooth modules, short range wireless components or protocols, or other wireless communication components and modules capable of sending and receiving electromagnetic signals. In this way, data from one device 102, 104, 106 of the system 100 can send and receive data via electromagnetic signals from any other device 102, 104, 106 of the system 100. In addition, each of the devices 102, 104, 106 shown as part of the system 100 in FIG. 1, as well as any other portable or wearable electronic device described herein, can also include one or more processors connected to the antenna of the device to process and interpret the information from the data received from one or more other devices in the system 100.

In addition, in at least one example, each of the devices 102, 104, 106 shown as part of the system 100 in FIG. 1, as well as any other portable or wearable electronic device described herein, can also include one or more motion sensors or other sensing modules. In one example, the sensing module can include an accelerometer. In one example, a sensing module of any of the devices described herein can include one or more gyroscopes. In any of the examples of portable and/or wearable electronic devices described herein, including devices 102, 104, and 106 of FIG. 1, each device is configured with one or more of these motion sensors such that the motion of each device 102, 104, 106 can be determined by a processor. Motion data detected by the sensors of the devices can be processed by the one or more processors of the devices to determine the motion, including a change in position and an acceleration, of any of the devices 102, 104, 106 of the system.

In at least one example, the head-mountable device 102 can include a motion sensor (not shown in FIG. 1) that detects a motion of the head-mountable device 102 (or detects/gathers motion data) and that motion data is transmitted via an antenna to one or more of the wearable electronic devices 104, 106. The head-mountable device 102 may or may not include a processor to process the data for interpreting the movement of the head-mountable device 102. In examples where the head-mountable device does not include a processor, an antenna of the head-mountable device 102 can send data associated with the motion detected by the sensor and a processor of either the electronic watch 104 and/or the smartphone 106 can process the data received by the antenna of that device. In examples where the head-mountable device 102 does include a processor connected to the motion sensor of the head-mountable device 102, the processor of the head-mountable device 102 can first process data associated with the detected motion of the head-mountable device 102 and then that processed information can be transmitted to one or more of the wearable electronic devices 104, 106 of the system 100.

Likewise, in at least one example, either of the wearable devices 104, 106, for example the electronic watch 104, can include a motion sensor (not shown in FIG. 1) that detects a motion of the electronic watch 104 (or detects/gathers motion data) and that motion data is transmitted via an antenna to the head-mountable device 102 or any other electronic device of the system 100. The electronic watch 104 may or may not include a processor to process the data for interpreting the movement of the electronic watch 104. In examples where the head-mountable device does not include a processor, an antenna of the electronic watch 104 can send data associated with the motion detected by the sensor and a processor of either the head-mountable device 102 and/or the smartphone 106 can process the data received by the antenna of that device. In examples where the electronic watch 104 does include a processor connected to the motion sensor of the electronic watch 104, the processor of the electronic watch 104 can first process data associated with the detected motion of the electronic watch 104 and then that processed information can be transmitted to one or more of the other devices 102, 106 of the system 100. The smartphone 106 or any other wearable or portable device of the system 100 can likewise be configured with one or more processors, antennas, and sensors to communicate detected motion with other devices of the system 100.

In this way, the system can include multiple electronic devices 102, 104, 106, both that detect motion and transmit data or information indicative or associated with the detected motions to one or more other devices 102, 104, 106 of the system 100. As noted above, the head-mountable device 102, electronic watch 104, and the smartphone 106 are exemplary only, and devices of the system 100 are not limited thereto. The system 100 can include only two devices, for example a head-mountable device 102 and an electronic watch 104, or the system 100 can include three or more devices including other wearable or portable electronic devices not shown in FIG. 1, or any combination thereof. In one example, the system can include a single earbud 102 and an electronic watch 104. In another example, the system 100 can include two earbuds 102 and an electronic watch 104 and/or smartphone 106.

In general, the devices of the system 100 are such that the motion detected by each device 102, 104, 106 indicates a motion of the part of the user 101 on which the device is worn or mounted. For example, the motion detected by the electronic watch 104 of the system 100 can be used to determine the motion of the user’s arm or wrist to which the electronic watch 104 secured. Likewise, the detected motion of head-mountable device 102 can be used to determine or estimate the motion of the user’s head to which the head-mountable device 102 is mounted. In one example, the determination of the motion of the user’s body part associated with a device can be a straightforward correlation to the device mounted to or worn on that part.

For example, the electronic watch 104 can be secured to the user’s wrist such that it does not move or only moves minimally relative to the user’s wrist during abrupt changes in acceleration of the user’s wrist. In such an example, the detected motion of the electronic watch itself will generally correlate to the actual motion of the user’s wrist. Likewise, the head-mountable device 102 can be mounted to the user’s head, for example at one or more of the user’s ears, such that the detected motion of the head-mountable device 102 correlates well with the actual motion of the user’s ears to which the head-mountable device 102 is mounted.

In at least one example, the motion of any of the devices 102, 104, 106 of the system can be processed to extrapolate the motion of another body part to which the device 102, 104, 106 is not secured but to which a relative position is known or can be estimated. For example, as noted above, the head-mountable device 102 can include one or more devices mounted to one or more ears of the user 101, for example an earbud. The earbud is located at the user’s ear. Based on anthropometric data in general, or based on specific anthropometric data of the user, a positon of the earbud 102 relative to another body part, for example the base of the neck of the user 101, can also be determined. In such an example, a motion of the base of the neck can be extrapolated from the detected motion of the earbud 102 based on the distance and known human anatomy of the neck and head. Thus, the head-mountable device 102 can be used to determine not only the motion of the head-mountable device 102 itself, but also the motions of various related or connected body parts of the user.

In one example, where the head-mountable device 102 includes two earbuds, with one earbud in each ear, the motion of each earbud fixed in position relative to one another in separate ears of the user 101 can be used in conjunction to extrapolate a three-dimensional motion of the user’s head and/or neck, including head rotation in any direction in space.

Likewise, the position of other body parts can be determined or estimated based on detected motions of any of the devices 102, 104, 106 of the system 100. For example, the detected motion of the electronic watch 104 can be extrapolated to determine the motion of the user’s forearm, elbow, or shoulder. In another example, detected motion of the smartphone 106 carried in the user’s pocket against the leg of the user 101 can be used to extrapolate the motion of the user’s knee or hip.

Certain detected motions, including an acceleration of the devices 102, 104, 106, can also be processed by one or more of the processors of the devices 102, 104, 106 of the system 100 to determine or estimate a force acting on an associated body part of the user 101. For example, a motion sensor of the head-mountable device 102 can detect an acceleration of the user’s head that indicates a certain force acting on the user’s head. One or more processors of the devices 102, 104, 106 of the system 100 can process the detected motion data and determine, based at least in part on the acceleration and extrapolated forces acting on the user’s head, whether an injurious event has occurred. An injurious event can be an event that includes motions of the user’s head and/or forces acting on the user’s head above a certain threshold. That is, the motion detected by the head-mountable device 102 can be processed to extrapolate and determine the forces or motions (including accelerations) experiences by the user’s head and neck. Because forces and accelerations that cause concussions or other brain, neck, or head injuries are known, the detected and extrapolated forces and accelerations can be compared to known injury thresholds to determine if and when the user 101 has experienced forces or accelerations that exceed a threshold and are potentially indicative of an injurious event such as a concussion or other head injury.

The same determination of forces or accelerations that exceed a threshold and are potentially indicative of an injurious event can be detected in like manner by any of the other devices 104, 106 of the system 100 to determine if other body parts associated therewith have also undergone threshold exceeding forces or accelerations that may be indicative of an injurious event. Other injurious events of other body parts can include whiplash events of the user’s neck or forces acting on the user’s arms, legs, hips, knees, or other parts and joints of the user that would tend to cause harm to those body parts.

The head-mountable device 102 shown in FIG. 1 can include one or more earbuds as noted above, but can also include other head-mountable devices 102 in one or more other examples. In at least one example, the head-mountable device can include a head-mountable display (HMD) as part of a head-mountable virtual/augmented reality device. In at least one example, the head-mountable device 102 can include electronic glasses. In at least one example, the head-mountable device 102 can include any other electronic device configured to be worn on or otherwise mounted to the head of the user 101.

Thus, as described above with reference to FIG. 1, an impact detection system 100 can include an earbud 102 having a first motion sensor and a wearable electronic device 104, 106 in electrical communication with the earbud 102. The wearable electronic device 104, 106 can include a second motion sensor electrically connected to a processor. In at least one example, the processor determines if an injurious event may have occurred by comparing a first motion detected by the first motion sensor to a second motion detected by the second motion sensor.

Any of the features, components, parts, including the arrangements and configurations thereof shown in FIG. 1 can be included, either alone or in any combination, in any of the other examples of devices, features, components, and parts shown in the other figures. Likewise, any of the features, components, parts, including the arrangements and configurations thereof shown in the other figures can be included, either alone or in any combination, in the example of the devices, features, components, and parts shown in FIG. 1.

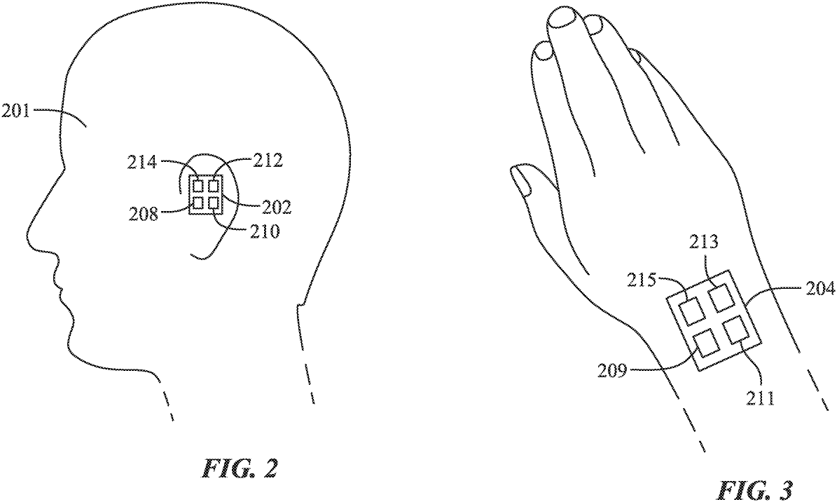

FIG. 2 shows an example of a head-mountable device 202 that can be a part of the systems described herein, including system 100 shown in FIG. 1 or systems shown in other figures. The head-mountable device 202 can be an earbud 202 mounted or secured to or at least partially within the ear of the user 201, as shown in FIG. 2. In at least one example, the earbud 201 can include a motion sensor 208, an antenna 210, and a processor 212 electrically coupled to both the antenna 210 and motion sensor 208. In at least one example, the earbud 202 can also include one or more additional electronic or other components 214, including, for example, one or more optical sensors, memory storage components, microphones, speakers, circuitry and circuitry components including printed circuit boards, housings, brackets, and the like.

As noted above, the motion sensor 208 of the earbud 202 is configured to detect a motion of the earbud 208. The processor 212, which can be electrically connected to the motion sensor 208, can receive and process data gathered or detected by the motion sensor 208 can determine a motion, including a velocity and acceleration of the earbud 202 as well as the head of the user 201. The term “motion,” as used herein, can also include a detected displacement or change in position or any combination of position, displacement, velocity, acceleration, direction or orientation of the user’s head, another body part, and/or the earbud 202. The processor 212, which is electrically connected to the antenna 210, can cause the antenna 210 to transmit the motion data or information to one or more other devices of the system. In at least one example, as noted above, the earbud 202 may not include a processor. Rather, the motion sensor 208 can be electrically connected to the antenna 210 such that data from the detected motion can be sent directly to another device of the system of which the earbud 202 is a part, for example another wearable electronic device of the system or another head-mountable device, such as another earbud, and one or more processors of the other device can process and interpret the motion data to determine whether threshold exceeding forces or accelerations that may be indicative of an injurious event have occurred.

In one example, another device of the system may be another earbud worn in the user’s other ear (not shown). The two earbuds can be configured to communicate via one or more antennas and processors, as described above, such that motion detected and data gathered at either earbud can be communicated to the other. In this way, motion can be detected at two distinct positions in space relative to the user’s head, for example at the positions of the user’s ears, so that more data can be gathered regarding the detected motion of the head and thus more information can be determined or processed regarding the three-dimensional movement or motion of the user’s head. For example, using two motion sensors of two separate earbuds, one at each of the user’s ears, the pitch, yaw, and roll of the user’s head can be determined as well as the overall acceleration of the head. Such motion data and information can be transmitted to one or more other devices of the system of which the earbuds are a part.

In at least one example, the earbud 202 can include one or more optical sensors 214, as noted above. The optical sensor 214 can be electrically connected to the processor 212 and can be configured to detect whether the earbud 202 is mounted to the user’s head, for example, to the user’s ear. In this way, when detecting and communicating the motion of the earbud 202, if the optical sensor 214 does not detect that the earbud 202 is being worn, any potentially injurious motions or events detected by the motion sensor 208 that exceed a predetermined threshold can be ignored or discounted. This may occur, for example, if the earbud 202 falls off the user’s head during use and hits the ground. The impact of the earbud 202 may register a detected potentially injurious motion via the motion sensor 208, but if the processor 212 determines, via the optical sensor 214, that the earbud 202 is not mounted to the user’s head at the time of the impact, the processor 212 can determine that the detected motion is not indicative of a motion of the user’s head. In such a case, the processor will not send a signal communicating the detected motion but may only send it when the earbud is being worn as detected by the optical sensor 214. In this way, the optical sensor 214 can reduce false injurious motions that may be detected when the earbud 202 is not in use.

In at least one example, as noted above, the earbud 202 can include a memory component 214 to store and record information regarding detected motions of the motion sensor 208. In this way, as the earbud 202 detects and communicates motions of the earbud 202, all or some of the data associated with the detected motions can be stored on the memory component 214 for later retrieval and communication within an impact detection system.

Any of the features, components, parts, including the arrangements and configurations thereof shown in FIG. 2 can be included, either alone or in any combination, in any of the other examples of devices, features, components, and parts shown in the other figures. Likewise, any of the features, components, parts, including the arrangements and configurations thereof shown in the other figures can be included, either alone or in any combination, in the example of the devices, features, components, and parts shown in FIG. 2.

FIG. 3 shows an example of a wearable electronic device 204 such as those described within systems shown in other figures. In the example of FIG. 3, the wearable electronic device 204 can be an electronic watch 204 worn on the wrist or arm of the user, as shown. In at least one example, the electronic watch 204 can include a motion sensor 209, an antenna 211, and a processor 213 electrically connected to the motion sensor 209 and the antenna 211. The electronic watch 204 can also include one or more other electronic components 215, for example, a display screen, memory component, microphone, speaker, circuitry and circuitry components including printed circuit boards, housings, brackets, or other structural or electronic components.

The electronic watch 204 can be one or multiple wearable and head-mountable devices within the systems described herein, including system 100 of FIG. 1 and systems shown in other figures. The antenna 211 of the electronic watch 211 can be configured as a receiver to receive transmitted motion data from one or more other devices of the system of which the electronic watch is a part. In at least one example, the electronic watch 204 receives motion data and information transmitted from an earbud, such as the earbud 202 shown in FIG. 2. The processors 213 of the electronic watch 204 can process the data or information received at the antenna 209 to determine whether threshold exceeding forces that may be indicative of an injurious event have occurred or been detected, for example whether the earbud 202 of FIG. 2, and thus, the user’s head to which the earbud is mounted, has undergone or experienced injurious forces or accelerations indicative of a potential injurious event.

Additionally, or alternatively, the motion sensor 209 of the electronic watch 204 can detect a motion of the electronic watch 204 itself and the processor 213 can determine if accelerations at the user’s wrist on which the electronic watch 204 is worn exceeds an injurious threshold acceleration. In this way, the electronic watch 204 can detect and determine whether the user’s body has potentially experienced an injurious event. If so, the processor 213 can cause the antenna 211 of the electronic watch 204 to transmit motion data or other signals to one or more other devices of the system, for example another wearable electronic device such as the smartphone 106 shown in FIG. 1 and/or the earbud 202 shown in FIG. 2.

Thus, as shown in FIG. 3, the electronic watch 204 can be a portable injury detection device that includes a motion sensor 209, a processor 213 electrically coupled to the motion sensor 209, and a receiver (such as an antenna) 211 configured to receive motion data from a head mountable device. The processor 213 can determine whether or not a potentially injurious motion of the head-mountable device has occurred by comparing data gathered by the motion sensor 209 with the motion data from the head-mountable device.

In at least one example, as noted above, the electronic watch 204 can include a memory component 215 to store and record information regarding detected motions of the motion sensor 209. In this way, as the electronic watch 204 detects and communicates motions of the electronic watch 204, all or some of the data associated with the detected motions can be stored on the memory component 215 for later retrieval and communication within an impact detection system.

Any of the features, components, parts, including the arrangements and configurations thereof shown in FIG. 3 can be included, either alone or in any combination, in any of the other examples of devices, features, components, and parts shown in the other figures. Likewise, any of the features, components, parts, including the arrangements and configurations thereof shown in the other figures can be included, either alone or in any combination, in the example of the devices, features, components, and parts shown in FIG. 3.

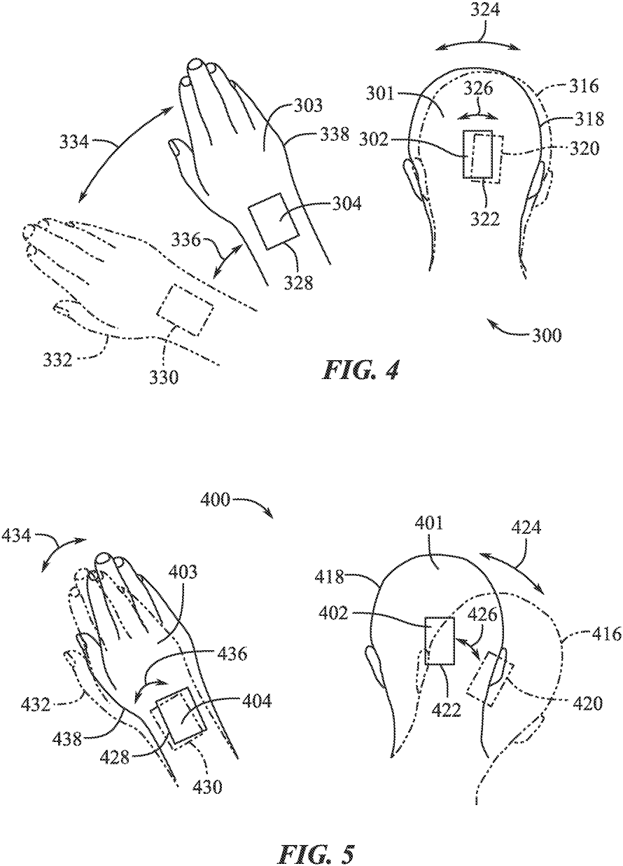

FIG. 4 illustrates another example of an impact detection system 300 including a head mountable device in the form of an earbud 302 mounted to the head of the user 301 and a wearable electronic device in the form of an electronic watch 304 worn on the wrist of the user 301. Similar to devices described herein as part of systems shown in other figures, the earbud 302 and electronic watch 304 are configured to detect motion and communicate with the other device regarding the detected motion to determine if an injurious event has potentially occurred.

As shown in FIG. 4, the user’s head to which the earbud 302 is mounted may move from a first head position 318 to a second head position 316. A head motion 324 associated with the movement of the head between the first and second positions 318, 316 is indicated by the arrows shown. As noted above, the head motion 324 can be extrapolated and determined from a corresponding earbud motion 326 indicative of a change in the earbud 302 from a first earbud position 322 to a second earbud position 320. For illustrative purposes, the head motion 324 and earbud motion 326 are shown as a difference in first and second positions 318, 316 and 322, 320, respectively. However, the head motion 324 and earbud motion 326 can include a change in position or velocity within a certain amount of time or a change in velocity over a certain amount of time such that both the head motion 324 and earbud motion 326 indicate an acceleration of the head 301 and the earbud 302, respectively. That is, while velocity over time indicating an acceleration is difficult to illustrate in a static, two-dimensional figure such as FIG. 4, the various head positions 316, 318 and earbud positions 320, 322 with corresponding head and earbud motions 324, 326 can also indicate acceleration and velocity magnitudes and events or occurrences. The term “motion,” as used herein, can also include a detected displacement or change in position or any combination of position, displacement, velocity, acceleration, direction or orientation of the user’s head, another body part, and/or the earbud 302.

Likewise, the electronic watch 304 can detect an electronic watch motion 336 based on a change in position of the electronic watch 304 from a first watch position 328 to a second watch position 330 over time. This watch motion 336 can be extrapolated to estimate or determine a hand motion 334 based on an extrapolated or estimated change in position of the user’s hand from a first-hand position 338 to a second hand position 332 over time. As noted above with reference to the head motion 324 and the earbud motion 326, the hand and watch motions 334, 336, as illustrated statically in FIG. 4, can be indicative and references as accelerations of the hand and electronic watch 304 shown.

As shown in FIG. 4, the watch and earbud motions 336, 326 may be different, indicating that the hand and head motions 334, 324 are different. For example, the acceleration experienced by the head of the user 301 may be smaller than an acceleration experienced by the hand of the user. In such a case, the user’s hand may experience accelerations over an injurious acceleration threshold but the user’s head may not. The various detected motions 324, 326, 334, 336 can be communicated between the earbud 302 and the electronic watch 304 and a processor can determine whether the user’s head has potentially experienced an injurious event or not. For example, a processor of the electronic watch 304 can determine that the watch motion 336 of the electronic watch 304, and thus the hand motion 334 of the user’s hand or wrist, has experienced an injury level acceleration. This may occur, for example, when the user falls down or hits a tree while skiing or biking. Once the system 300 has determined that threshold exceeding forces or accelerations that may be indicative of an injurious motion or event have occurred to the electronic watch 304 and/or hand or arm of the user, the processor of the electronic watch 304 can cause the antenna of the watch 304 to communicate with the other devices of the system, for example the earbud 302, and receive motion data from the earbud 302. The earbud motion 324, in one example, may include acceleration below an injurious threshold such that the processor of the electronic watch 304 can compare the head and/or earbud motion 324, 326 to determine whether the user’s head 301 has experienced a potentially injurious motion or event.

Any of the features, components, parts, including the arrangements and configurations thereof shown in FIG. 4 can be included, either alone or in any combination, in any of the other examples of devices, features, components, and parts shown in the other figures. Likewise, any of the features, components, parts, including the arrangements and configurations thereof shown in the other figures can be included, either alone or in any combination, in the example of the devices, features, components, and parts shown in FIG. 4.

FIG. 5 illustrates another example of an impact detection system 400 including a head mountable device in the form of an earbud 402 mounted to the head of the user 401 and a wearable electronic device in the form of an electronic watch 404 worn on the wrist of the user 401. Similar to devices described herein as part of systems shown in other figures, the earbud 402 and electronic watch 404 are configured to detect motion and to communicate with the other device regarding the detected motion to determine if an injurious event has potentially occurred.

As shown in FIG. 5, the user’s head to which the earbud 402 is mounted may move from a first head position 418 to a second head position 416. A head motion 424 associated with the movement of the head between the first and second positions 418, 416 is indicated by the arrows shown. As noted above, the head motion 424 can be extrapolated and determined from a corresponding earbud motion 426 indicative of a change in the earbud 402 from a first earbud position 422 to a second earbud position 420. For illustrative purposes, the head motion 424 and earbud motion 426 are shown as a difference in first and second positions 418, 416 and 422, 420, respectively. However, the head motion 424 and earbud motion 426 can include a change in position or velocity within a certain amount of time or a change in velocity over a certain amount of time such that both the head motion 424 and earbud motion 426 indicate an acceleration of the head 401 and the earbud 402, respectively. That is, while velocity over time indicating an acceleration is difficult to illustrate in a static, two-dimensional figure such as FIG. 4, the various head positions 416, 418 and earbud positions 420, 422 with corresponding head and earbud motions 424, 426 can also indicate acceleration and velocity magnitudes and events or occurrences. The term “motion,” as used herein, can also include a displacement or change in position or any combination of position, displacement, velocity, acceleration, direction, or orientation of the user’s head, another body part, and/or the earbud 402.

Likewise, the electronic watch 404 can detect an electronic watch motion 436 based on a change in position of the electronic watch 404 from a first watch position 428 to a second watch position 430 over time. This watch motion 436 can be extrapolated to estimate or determine a hand motion 434 based on an extrapolated or estimated change in position of the user’s hand from a first-hand position 438 to a second hand position 432 over time. As noted above with reference to the head motion 424 and the earbud motion 426, the hand and watch motions 434, 436, as illustrated statically in FIG. 4, can be indicative and references as accelerations of the hand and electronic watch 404 shown.

As shown in FIG. 5, the watch and earbud motions 436, 426 may be different, indicating that the hand and head motions 434, 424 are different. For example, the acceleration experienced by the head of the user 401 may be larger than an acceleration experienced by the hand or arm of the user. In such a case, the user’s head may experience accelerations over an injurious acceleration threshold but the user’s hand or arm may not. The various detected motions 424, 426, 434, 436 can be communicated between the earbud 402 and the electronic watch 404, and a processor can determine whether the user’s head has likely experienced an injurious event or not.

For example, a processor of the electronic watch 404 can determine that the earbud motion 426 has risen to a potentially injurious level of acceleration or motion. This may occur, for example, when the user falls, is in an automobile accident, or hits a tree while skiing or biking. However, in some cases as shown in FIG. 5, where the watch motion 436 and thus the hand motion 434 is less than a potentially injurious acceleration threshold or different from the head motion 424, the detected injurious earbud and head motion 426, 424 can trigger a processor of the earbud 402 and/or electronic watch 404 to determine whether the user has experienced a potentially injurious event at the head or arm.

Once the system 400 has determined that a potentially injurious motion or event has occurred to the earbud 402 and/or head of the user, the processor of the watch 404 and/or earbud 402 can cause the antenna of the watch 404 and/or earbud 402 to communicate with the other devices of the system, for example, another earbud or wearable electronic device, and receive motion data from the various devices to confirm an accurate motion of the user.

Any of the features, components, parts, including the arrangements and configurations thereof shown in FIG. 5 can be included, either alone or in any combination, in any of the other examples of devices, features, components, and parts shown in the other figures. Likewise, any of the features, components, parts, including the arrangements and configurations thereof shown in the other figures can be included, either alone or in any combination, in the example of the devices, features, components, and parts shown in FIG. 5.

In some examples, as the user moves and is active, for example while riding a bicycle or jogging, lifting weights, skiing, or the like, the motions of each device of the systems described herein can be detected and communicated to other devices within the system, as noted above. If a potentially injurious event is determined by detected motion of one or more devices but not at one or more other devices a processor of one of the devices of the system can determine whether a potentially injurious event has occurred or been experienced at one or more body parts of the user by comparing the motion detected at one device with motion detected at another device.

For example, the user can secure the head-mountable device, such as one or more earbuds, to the user’s head and secure the electronic watch to the user’s wrist during skiing. Motion at both the head-mountable device and the electronic watch can be detected and processed to determine if either body part, meaning the head or the wrist, associated with the devices has experienced a potentially injurious event. In some cases, both devices will detect a force or acceleration indicative of an injurious event. In other cases, only one of the devices will detect a potentially injurious event. In cases where only one device detects the potentially injurious event, depending on which device detects the potentially injurious event and what motion or forces are detected and extrapolated from the other device the motion data of the devices can be compared with each other by one or more processors of either of the devices to determine whether the user has likely experienced an injurious event or is likely to have an injury.

In general, FIG. 4 and FIG. 5 illustrate two scenarios in which separate electronic devices of a system may experience different motions, including different acceleration magnitudes during use. In some examples, both devices of the systems shown in FIGS. 4 and 5 can experience threshold exceeding forces or accelerations that may be indicative of injurious events or motions exceeding an injurious threshold motion. In such a case, one or more processors of the devices in the system can determine that the user has potentially experienced an injury or at least an injurious event or motion has occurred. FIG. 4 illustrates an example scenario where a user may experience motions including accelerations above an injurious threshold at one body part but not the other.

In such a scenario as shown and described with reference to FIG. 4, while the electronic watch 304 may experience or detect threshold exceeding forces or accelerations that may be indicative of an injurious event, this doesn’t always mean that the user’s head has also experienced an injurious event. As described above, the earbud 302 may not detect threshold exceeding forces or accelerations that may be indicative of an injurious event at the user’s head. This may be the case, for example, if the user’s hand hits a tree while skiing down a hill but the user’s head does not. This may also be the case, for example, when the user is striking a punching bag during a workout. The hand of the user may repeatedly experience threshold exceeding forces or accelerations that may be indicative of an injurious event while the user’s head remains safe. Many other scenarios can be conceived in which the wearable device of the system, whether it be worn on the user’s wrist, in the user’s pocket, or elsewhere, detects a potentially injurious motion but the user has not experienced an injurious event at the head.

Accordingly, the multiple devices of impact detection systems described herein can increase the accuracy of concussive event detection by providing multiple points of reference from which motion data can be detected and communicated to accurately assess a potentially injurious event. Any one of the devices of impact detection systems described herein can instigate or initiate an injury detection protocol or alert depending on which device detects a potentially injurious event.

Conversely to FIG. 4, FIG. 5 illustrates a case or scenario where a user may experience motions including accelerations above an injurious threshold at the user’s head, but not at one or more other body parts. In such a case, one or more wearable device of the system, such as the electronic watch 404, may not detect threshold exceeding forces or accelerations that may be indicative of an injurious event while the head-mountable device, such as the earbud 402, does detect threshold exceeding forces or accelerations that may be indicative of an injurious event. In this example, even though the wearable device does not detect an injurious event, motion data detected by the head-mountable device can be communicated to one or more devices and one or more processors of the one or more devices of the system can determine that an injurious event may have occurred at the user’s head.

In at least one example, the impact detection systems described herein can include one or more user feedback mechanisms to alert or otherwise communicate with the user when a potentially injurious event has occurred, especially when the system has determined that the user’s head may have experienced an injury. Each electronic device of the system, including the head-mountable devices and the various wearable electronic devices, can include one or more feedback components, including one or more visual display screens, audio output speakers, and/or tactile feedback motors and mechanisms. When a potential injury is detected, one or more of the feedback mechanisms can be activated to communicate with the user.

In one example, audio output through the earbuds of the system can announce that a possible injury event has been detected. A programmed voice can request that the user respond to confirm or deny that the concussive event has taken place. If the user does not respond or confirms that the injurious event has taken place, one or more safety protocols can be put into effect. Likewise, the watch or other wearable electronic device of the systems disclosed herein can visually display a warning or the detection of the injurious event.

In at least one example, when any of the electronic devices of systems described herein individually or collectively detect and determine that an injurious event has likely occurred, the one or more signals communicated by the devices of the system can include alerts, warnings, or emergency contact functions and protocols. For example, absent any interceding action or reply from the user to an injurious event detection and alert of the system, one or more processors of the wearable electronic device, for example a smartphone or electronic watch, can automatically call an emergency number to notify emergency and medical personal nearby of the potentially injurious event.

In at least one example, once a potentially injurious event or motion has been detected by one or more of the systems described herein, one or more devices of a system, including the head-mountable and wearable devices described herein, can continue sensing, monitoring, recording, and/or communicating detected motions of the devices. Motions detected after the system has determined an injurious event has potentially occurred can be indicative of the status of a user who has undergone a potentially injurious event. For example, if no motion has been detected for a certain period of time after a potentially injurious event has been detected, the lack of motion may indicate that the user is potentially unconscious from the detected impact or fall. In another example, the wearable electronic device can be configured to monitor heart rate or breathing including oxygen levels of the user after a fall or potentially injurious impact event has been detected. In such cases, one or more emergency communications or protocols can be performed by the system to seek help.

Additionally, in at least one example, one or more processors of the one or more impact detection systems described herein can use a device’s location as another input to consider when determining if a potentially injurious event has likely occurred. For example, if the user’s location is a ski resort or bike trail, this can be considered in determining whether an injurious event has likely occurred. Likewise, one or more other software applications of the devices can be used to determine a user’s activity or location to better determine whether an injurious event has occurred. For example, the user’s calendar software application on an electronic watch or smartphone may indicate to one or more processors of the system that at the time of the detected potentially injurious motion or event, the user was scheduled to be at the boxing gym working out with his or her trainer. This type of information can be taken into account to determine whether the injurious event is more or less likely to have occurred when detected.

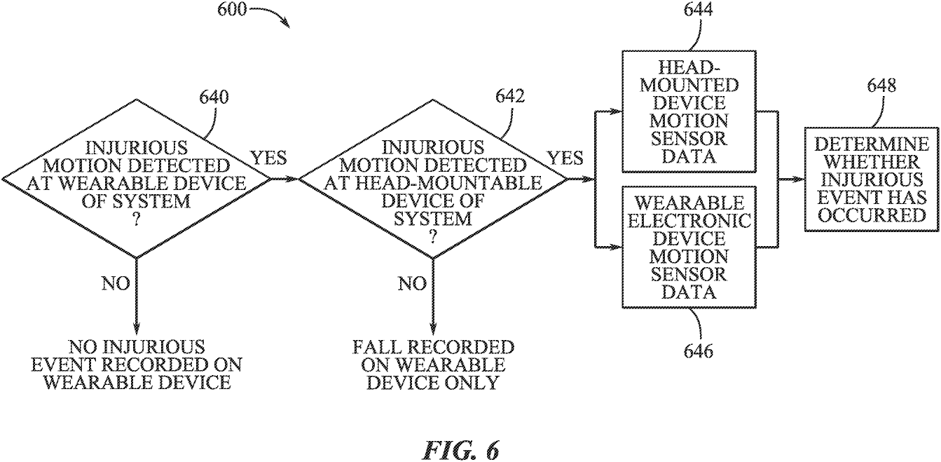

FIG. 6 shows a flowchart of a method 600 of detecting a potential injury using the impact detection systems described herein. As shown at 640, the method 600 can include determining whether a potentially injurious motion has been detected at a wearable electronic device of an impact detection system, including systems described herein. If no potentially injurious motion is detected by the wearable electronic device, no injurious event is recorded or communicated by the wearable electronic device. If a potentially injurious motion or event that exceeds a predefined threshold is detected by the wearable device, the impact detection system can determine, at 642, if a potentially injurious motion or event has been detected at a head-mountable device of the system.

At 642 of method 600, if no potentially injurious motion or event is detected by the head-mountable device of the system, the fall or injurious event or motion can be recorded and/or communicated only on the wearable electronic device. If, at 642, a potentially injurious motion or event is detected by or at the head-mountable device of the system, information and motion data detected by one or more motion sensors of both the head-mountable device at 644 and the wearable electronic device at 646 can be used to determine whether a potentially injurious event has occurred at 648.

In the method 600 shown in FIG. 6 and described above, determining whether or not threshold exceeding forces that may be indicative of an injurious motion or event have occurred can begin with or be instigated by the wearable electronic device of the impact detection system. In such an example, the head-mountable device of the impact detection system can function to corroborate or confirm the potentially injurious motion or event detected by the wearable device. In this way, the head-mountable device can increase the accuracy of injurious event detection and determination of the system as opposed to only relying on the wearable electronic device to detect potential user falls and injuries. Along these lines, method 600 can include detecting motions, determining whether potentially injurious events occur, and communicating motion data using more than one head-mountable device and one wearable device. An increased number of devices in the system can increase the number of data sources used in the injurious event determination in order to increase the accuracy of identifying when a user experiences a potentially injurious event.

Any of the features, components, parts, steps, or alternatives suggested with reference to the method shown in FIG. 6 can be included, either alone or in any combination, in any of the other examples of methods shown in the other figures. Likewise, any of the features, components, parts, steps, or alternatives suggested with reference to the method shown in the other figures can be included, either alone or in any combination, in the example of the method shown in FIG. 6.

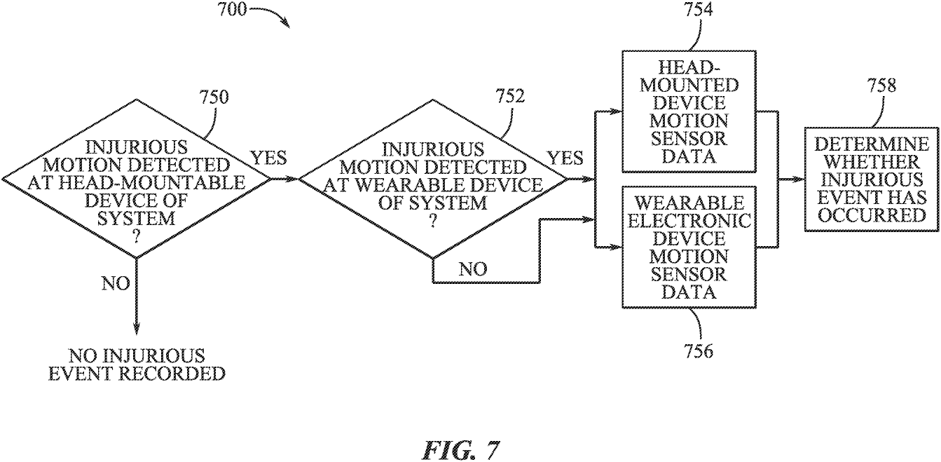

FIG. 7 shows a flowchart of a method 700 of detecting a potential injury using the impact detection systems described herein. As shown at 750, the method 700 can include determining whether a potentially injurious motion has been detected at a head-mountable device of an impact detection system, including systems described herein. If no potentially injurious motion is detected by the head-mountable device, no injurious event is recorded or communicated by the head-mountable device. If a potentially injurious motion or event is detected by the head-mountable device, the impact detection system can determine, at 752, if a potentially injurious motion or event has been detected at a wearable device of the system.

At 752 of method 700, if no potentially injurious motion or event is detected by the wearable electronic device of the system, the fall or potentially injurious event or motion can be recorded and/or communicated on the head-mountable device. If, at 752, a potentially injurious motion or event is detected by or at the head-mountable device of the system, information and motion data detected by one or more motion sensors of both the head-mountable device at 754 and the wearable electronic device at 756 can be used to determine whether a potentially injurious event has occurred at 758. In addition, in examples where no potentially injurious motion or event is detected by the wearable electronic device of the system, motion data can still be communicated at 756 with other devices of the system, including the head-mountable device at 754, to determine whether a potentially injurious event has occurred at 758.

In the method 700 shown in FIG. 7 and described above, determining whether or not a potentially injurious motion or event has occurred can begin with or be instigated by the head-mountable device of the impact detection system. In such an example, the wearable electronic device of the impact detection system can function to corroborate or confirm the concussive motion or event detected by the head-mountable device. In this way, the wearable device can increase the accuracy of injurious event detection and determination of the system as opposed to only relying on the head-mountable device to detect user falls and injuries. Along these lines, method 700 can include detecting motions, determining whether potentially injurious events occur, and communicating motion data using more than one head-mountable device and one wearable device. An increased number of devices in the system can increase the number of data sources used in the injurious event determination in order to increase the accuracy of identifying when a user experiences an injurious event.

Any of the features, components, parts, steps, or alternatives suggested with reference to the method shown in FIG. 7 can be included, either alone or in any combination, in any of the other examples of methods shown in the other figures. Likewise, any of the features, components, parts, steps, or alternatives suggested with reference to the method shown in the other figures can be included, either alone or in any combination, in the example of the method shown in FIG. 7.

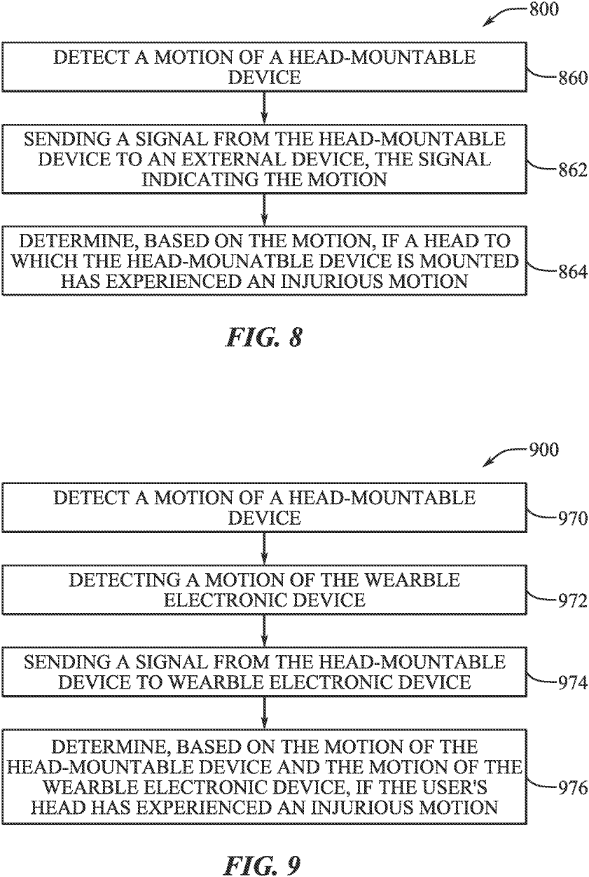

FIG. 8 shows a flowchart of a method 800 of detecting a potential injury. At 860, a motion of a head-mountable device can be detected. At 862, a signal can be sent from the head-mountable device to an external device, the signal indicating the motion. In at least one example, the external device can be one or more of the wearable electronic devices described herein and shown in the figures, for example an electronic watch or a smartphone. At 864, the method 800 includes determining, based on the detected and sent/communicated motion, if a user’s head to which the head-mountable device is mounted has experienced threshold exceeding forces or accelerations that may be indicative of injurious motion.

Any of the features, components, parts, steps, or alternatives suggested with reference to the method shown in FIG. 8 can be included, either alone or in any combination, in any of the other examples of methods shown in the other figures. Likewise, any of the features, components, parts, steps, or alternatives suggested with reference to the method shown in the other figures can be included, either alone or in any combination, in the example of the method shown in FIG. 8.

FIG. 9 shows a flowchart of a method 900 of detecting a potential injury. At 970 of the method 900, an impact detection system can detect a motion of a head-mountable device and detect a motion if the wearable electronic device at 972. At 974 of the method 900, the system can send a signal from the head-mountable device to the wearable electronic device. At 976 of the method 900, the system can determine, based on the motion of the head-mountable device and the motion of the wearable electronic device, if the user’s head has experienced threshold exceeding forces or accelerations that may be indicative of injurious motion.

Any of the features, components, parts, steps, or alternatives suggested with reference to the method shown in FIG. 9 can be included, either alone or in any combination, in any of the other examples of methods shown in the other figures. Likewise, any of the features, components, parts, steps, or alternatives suggested with reference to the method shown in the other figures can be included, either alone or in any combination, in the example of the method shown in FIG. 9.

In at least one example of a method of detecting a potential injury, for example methods 800 and 900 shown in FIGS. 8 and 9, respectively, before determining the head has experienced a potentially injurious motion, the system can be configured to determine an acceleration of the head based on the detected motion of the head-mountable device. Additionally, in at least one example of a method of detecting a potential injury, for example methods 800 and 900 shown in FIGS. 8 and 9, respectively, before determining the acceleration of the head, the system can detect whether the head-mountable device is mounted to the head. This can be done as noted above, for example, using one or more optical sensors of the head-mountable device electrically connected to a processor.

To the extent applicable to the present technology, gathering and use of data available from various sources can be used to improve the delivery to users of invitational content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, TWITTER® ID’s, home addresses, data or records relating to a user’s health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user’s general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user’s privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not target to be exhaustive or to limit the embodiments to the precise forms disclosed. It will be apparent to one of ordinary skill in the art that many modifications and variations are possible in view of the above teachings.

What is claimed is:

1. A portable impact detection device, comprising:
a first motion sensor to gather first motion data associated with the portable impact detection device;
a first processor electrically coupled to the first motion sensor; and
a receiver configured to receive second motion data from a head-mountable device;
wherein the first processor determines that a threshold motion of the head-mountable device has occurred based on the first motion data and the second motion data.

2. The portable impact detection device of claim 1, wherein the head-mountable device comprises an earbud, the earbud comprising:
a second motion sensor;
an antenna; and
a second processor electrically connected to the second motion sensor and the antenna, the second processor identifying a threshold motion detected by the second motion sensor and causing the antenna to send a signal comprising the second motion data.

3. The portable impact detection device of claim 2, wherein the signal is sent to the portable impact detection device.

4. The portable impact detection device of claim 1, wherein the portable impact detection device comprises a wearable electronic device.

5. The portable impact detection device of claim 4, wherein the wearable electronic device comprises a smartwatch.

6. The portable impact detection device of claim 4, wherein the wearable electronic device comprises a smartphone.

7. A motion detection system, comprising:
an earbud including a first motion sensor; and
a wearable electronic device in electrical communication with the earbud and comprising a second motion sensor electrically connected to a processor;
wherein the processor determines if a threshold event has occurred based on a first motion detected by the first motion sensor and a second motion detected by the second motion sensor.

8. The motion detection system of claim 7, the earbud further comprising an antenna electrically connected to the first motion sensor, the antenna configured to transmit data associated with the first motion to the wearable electronic device.

9. The impact detection system of claim 7, wherein the earbud further comprises an optical sensor configured to detect when the earbud is mounted to a user.

10. The motion detection system of claim 9, wherein the processor causes the antenna to transmit the data if the optical sensor detects the earbud mounted to the user.

11. The motion detection system of claim 8, wherein the data indicates an acceleration of a head of a user to which the earbud is mounted.

12. The motion detection system of claim 7, wherein the wearable electronic device comprises an electronic watch.

13. The motion detection system of claim 7, wherein the wearable electronic device comprises a smartphone.

14. The motion detection system of claim 7, wherein:
the head-mountable device is a first head-mountable device and the motion detection system further comprises a second head-mountable device having a third motion sensor; and
the processor determines if the threshold event has occurred based on a first motion detected by the first motion sensor, a second motion detected by the second motion sensor, and a third motion detected by the third motion sensor.

15. A method of detecting a threshold motion, comprising:
detecting a motion of a head-mountable device;
sending a signal from the head-mountable device to an external device, the signal indicating the motion; and
determining, based on the motion, a head to which the head-mountable device is mounted has experienced the threshold motion.

16. The method of claim 15, wherein the external device comprises a wearable electronic device.

17. The method of claim 16, further comprising detecting a motion of the wearable electronic device.

18. The method of claim 17, wherein determining the head has experienced the threshold motion is based on the motion of the head-mountable device and the motion of the wearable electronic device.

19. The method of claim 15, further comprising determining an acceleration of the head based on the detected motion of the head-mountable device.

20. The method of claim 19, further comprising detecting when the head-mountable device is mounted to the head.
